(19) United States
(12) Patent Application Publication
(10) Pub. No.: US 2004/0038863 A1
(43) Pub. Date: Feb. 26, 2004

(54) Medical indication

(76) Inventor: Barbara Lippe, Los Angeles, CA (US)

Correspondence Address: DINSMORE & SHOHL, LLP, 1900 CHEMED CENTER, 255 EAST FIFTH STREET, CINCINNATI, OH, 45202, US

(21) Appl. No.: 10/457,649

(22) Filed: Jun. 9, 2003

Related U.S. Application Data
(60) Provisional application No. 60/388,562, filed on Jun. 13, 2002.

(51) Int. Cl. A61K 38/27; A61K 31/56

(52) U.S. Cl. 514/2; 514/171

(57) ABSTRACT

An aromatase inhibitor, alone or in combination with Growth Hormone, is used in the treatment of growth failure/short stature and in order to improve final height. It is believed that the present treatments delay bone maturation, especially in the treatment of pre-peri and pubertal humans with open epiphyses. The treatment is especially suitable for boys, with or without growth hormone deficiency (GHD).

### RELATED APPLICATION

The present application claims priority under 35 U.S.C. §119 of U.S. application Ser. No. 60/388,562 filed Jun. 13, 2002.

### FIELD OF THE INVENTION

The invention relates methods which employ an aromatase inhibitor, alone or in combination with Growth Hormone, in the treatment of growth failure/short stature and in order to improve final height. The invention further relates to such methods wherein delay of bone maturation is achieved, for example, in the treatment of preperi and pubertal humans with open epiphyses. The treatment is preferably for boys, with or without growth hormone deficiency (GHD). The invention further relates to the use of aromatase inhibitor in such treatments and to medicaments for use in such treatments.

### BACKGROUND

Aromatase is an enzyme that catalyzes the aromatization of C₁₉ androgens (androstenedione and testosterone) to obtain C₁₈ estrogens (estrone and estradiol).

Exemestane, (6-methylenandrosta-1,4-diene-3,17-dione), commercially available as Aromasin®, is an irreversible inhibitor of aromatase. Exemestane is structurally related to the natural substrate androstenedione, and has irreversible inhibitory properties on the aromatase enzyme.

In Growth Hormone (GH) deficient children, growth hormone is well established in the treatment of growth failure. However, the close correlation between the benefit of GH therapy and bone age (5) makes the use of GH less efficacious during puberty since the time window is limited by epiphyseal fusion. A limiting factor of growth failure therapy with GH is thus the closure of the growth plate in adolescence.

There are a large number of short children best classified as idiopathic short stature (ISS) with no identifiable cause for their growth retardation and for these children there are no proven good treatments to increase ultimate height. GH has been used extensively in this population, but with mixed and modest results (10).

Other approaches to treat short children have been suggested, such as GH treatment in addition to pubertal suppression with the administration of Gonadotropin (Gn)-releasing hormone (RH) analogues. However, the treatment of pubertal short children with GnRH analogues raises important issues pertaining to bone health and physiological well being as these children are rendered sexually infantile at a critical time of development. Pharmacological suppression by GnRH depletes the patient of all sex hormones—a treatment which is established in children with growth failure.

There are recent findings that estrogens are a key regulator of growth and that estrogens play the key role in promoting bone maturation and the closure of the growth plate in both genders. The role of endogenous estrogens in the control of GH secretion has been investigated by D. Metzger et al (7).

It is known that aromatase inhibitors selectively suppress estrogen levels, menstruation, growth rate and bone maturation rate in girls (6).

At the Pediatric Research Meetings in May 1999, Dr. Nelly Mauras, (11) US, reported a study on the metabolic effects of selective estrogen suppression in young males utilizing a potent and selective non-steroidal aromatase inhibitor, Anastrozole (Arimidex®, Zeneca Pharmaceuticals). See also, Mauras, N., et al, “Estrogen Suppression in Males: Metabolic Effects,” J. Clin. Endocrinol. Metab., 85:2379-77 (2000).

The use of Gonadotropin-Releasing Hormone agonist and inhibitors of the enzyme aromatase has been suggested for treatment of GHD (9).

The use of aromatase inhibitors has earlier been limited to pubertal children and no experiences have been reported from the prepubertal period.

Knowledge of the physiological role of estrogens in the regulation of growth derives from studying patients with partial or complete estrogen deficiency and patients with end-organ resistance to estrogens due to mutations within the cytochrome P450 aromatase gene (CYP19 gene) and the estrogen receptor (ER), respectively (1,2). Cytochrome P450 aromatase is essential for synthesis of estrone and estradiol from the androgen precursors androstenedione and testosterone. Inhibition of the cytochrome P450 aromatase therefore leads to a complete suppression of endogenous estrogens.

The specific action of cytochrome P450 aromatase inhibitors prevents the closure of the growth plate and decreases bone age advancement, thereby eliminating this restriction.

Characteristics of patients with cytochrome P450 deficiency and estrogen receptor mutations are summarized as follows:

Female; no thelarche/menarche, virilization, lack of growth spurt, delayed bone age and continued growth in adulthood, changes in lipid metabolism, hyperinsulinemia, and osteoporosis.

Male; same profile except normal sexual development.

Regardless of the genotype, all these patients present have symptoms related to an estrogen deficiency. In contrast to females, male patients show a normal sexual development with complete virilisation and masculinisation. Despite their lack of pubertal growth spurt, they reach a markedly increased final height (>200 cm) due to longer duration of the growth period caused by retardation of the bone age (maturation) and delayed closure of the growth plate. Both sexes develop severe osteopenia and consequently osteoporosis from the period of being estrogen deficient present from birth and continued until the time of ascertainment. In addition, “the aromatase excess syndrome” is associated with feminization of both sexes (3).

Aromatase inhibitors have been developed for advanced breast cancer therapy in postmenopausal women (4) and their use has been restricted only to the field of gynecology/oncology. Recently developed products (e.g. Letrozole) are cytochrome P450 aromatase specific and do not interact with the glucocorticoid-mineralcorticoid metabolism. Moreover they combine superior efficacy with better tolerability.

There is a need for possible treatment of short prepubertal, peripubertal and pubertal children, who cannot benefit sufficiently from growth hormone treatment or other suggested treatments.

### SUMMARY OF THE INVENTION

The present growth failure therapy with aromatase inhibitor, alone or together with growth hormone (GH), for the first time allows treatment of growth failure, even in patients during puberty, adolescence and early adulthood.

Males in contrast to female individuals with aromatase deficiency are not impaired in their gender specific sexual development. Therefore, a therapy with aromatase inhibitor, for example cytochrome P450 aromatase inhibitor, is expected to be most beneficial for male patients although female patients may benefit if virilization does not occur.

Aromatase inhibitors are known and include, but are not limited to the following compounds: exemestane, formestane, atamestane, fadrozole, letrozole, vorozole and anastrozole.

In specific embodiments of the invention, the aromatase inhibitor may comprise exemestane, anastrozole or letrozole. In a further embodiment, the aromatase inhibitor comprises exemestane. The aromatase inhibitor is administered in an amount effective to improve final height. For example, in one embodiment, the aromatase inhibitor may be administered in an amount of from about 1 to about 200 mg/day or from about 50 to about 500 mg/injection. In more specific embodiments, the following dosages are exemplary:

Exemestane: orally in a dosage range from about 5 to about 200 mg/day, preferably from about 10 to about 50 mg/day, more preferably from about 10 to about 25 mg/day, or parenterally in a dosage range of from about 50 to 500 mg per injection.

Fadrozole: orally in a dosage range from about 0.5 to about 10 mg/day, preferably from about 1 to about 2 mg/day.

Letrozole: orally in a dosage range from about 0.5 to about 10 mg/day, preferably from about 1 to about 2.5 mg/day.

Formestane: parenterally in a dosage range from about 250 to about 500 mg, preferably from about 250 to about 300 mg, per injection.

Anastrazole: orally in a dosage range from about 0.5 to about 10 mg/day, preferably from about 1 to about 2 mg/day.

In one embodiment, the treatment employs a combination of aromatase inhibitor and GH. Preferably human GH is used and the dose administered is from about 0.01 to about 0.1 mg/kg/day, more preferably from about 0.02 to about 0.1 mg/kg/day, further preferred from about 0.04 to about 0.1 mg/kg/day. In specific embodiments, GH doses of from about 0.026 to about 0.034, from about 0.034 to about 0.043 or from about 0.043 to about 0.1 mg/kg/day are used.

In the combination therapy, the aromatase inhibitor is preferably administrated simultaneous with the growth hormone, but the compounds can also be administered separately, for example at spaced intervals or sequentially. The GH may be wild type or recombinant GH, or an analogue thereof which exhibits GH activity, i.e., binds to GH receptor. The aromatase inhibitor is employed for increasing final height of children, particularly boys, with ISS. The use of the aromatase inhibitor in children with short stature—ISS or GHD—will offer pediatric endocrinologists a clinical tool to optimize linear growth in children already on GH therapy. The opportunity of providing pediatric endocrinologists with this treatment may also capture new and novel patients with either ISS or GHD.

The following exemplary groups of patients can be treated with the aromatase inhibitor or a combination of aromatase inhibitor and GH.

Prepubertal or peripubertal children with impaired growth, short stature (GHD and ISS) and not yet completed closure of the growth plates—delay of puberty.

Short stature children when it is desired to delay epiphysial closure and allow more time for GH promoted bone linear growth.

Pubertal children (adolescence and early adulthood if epiphysis are not yet fused) with impaired growth (GHD and ISS)

Male patients in puberty.

Adolescent and young adult male subjects if epiphysis are not yet fused.

Hypogonadotropic hypogonadism in adolescents if epiphyses are not yet fused (in conjunction with testosterone therapy to induce puberty but aromatase inhibition to keep epiphyses open).

Other individuals suitable for treatment within the scope of the invention will be apparent to one of ordinary skill in the art in view of the present specification.

In one embodiment, the methods of the invention comprise administration of an aromatase inhibitor, alone or in combination with Growth Hormone, to children, especially with idiopathic short stature, constitutional growth delay, or growth hormone deficient low status. Evidence exists that even in males the closure of the epiphysis (and the consequent stop of growth of bones in length) is mainly under the control of estrogens. Thus, without intending to be bound by theory, treatment with an aromatase inhibitor is believed to delay the closure of the epiphysis and allow more time for GH therapy, therefore allowing children to reach a higher stature.

In the examples below, exemestane, commercially available as Aromasin®, is used as aromatase inhibitor and recombinant human Growth Hormone, commercially available as Genotropin®, is used as recombinant growth hormone

### EXAMPLE 1

Males entering normal spontaneous puberty over the age of 11 years, having a bone age, using Greulich and Pyle Standard Atlas (12) of less than 15.5 years, and having a predicted final height of less than 1 SD from mid parental target height or a predicted height <1 SD from population mean I (using the tables for predicting adult height of Bayley and Pinneau developed for the Greulich and Pyle atlas) are studied in this example.

Dosage Form and Regime

Aromasin® tablets of 25 mg strength are used.

Part 1 of the study (open label, single center):

Two different doses (25 and 50 mg daily) of exemestane are administered orally for 10 days with a 14 day washout in between in random order. The washout period was selected as seven times the terminal elimination half-life of the drug (approximately 24 hours in postmenopausal women), plus 7 extra days to allow for the recovery of the aromatase enzyme. A group of 12 normal young males (ages 14-25 years), who have normal pubertal history and are at least Tanner stage IV-V of genital development is recruited. Either short or normally statured children are included.

Study Endpoints

1) Safety;

2) Efficacy in suppression of estradiol and Pharmacodynamics (circulating levels of estradiol, estrone, testosterone, IGF-1, IGFBP-3, SHBG, and DHEAS;

3) Pharmacokinetic parameters.

Part 2 of the study:

The PK study is performed in a group of 10 late pubertal or young men (14-25) after a single oral dose of exemestane. The dose is chosen based on the data obtained in part 1. Blood is withdrawn at: 0, 1, 2, 3, 4, 8, 12, 24, 48, 72, 120, and 240 hours after the administration of exemestane.

Study Endpoints

1) Safety;

2) Pharmacodynamics (circulating levels of estradiol, estrone, testosterone, IGF-1, IGFBP-3, SHBG, and DHEAS);

3) Pharmacokinetic parameters.

This study determines optimum doses of exemestane to lower estradiol concentrations in pubertal adolescent males with short stature in order to prolong the length of the growth phase, so that predicted final height improves, while preserving pubertal growth and physical virilization.

### EXAMPLE 2

Patient Population

A total of 100 adolescent males is entered.

Study Endpoint

The study is a 12-month double blind, placebo controlled trial.

Endpoint:

Change in predicted adult height for treated vs. placebo group. Analysis by both group data and paired “t” analyses.

Dosage Form and Regime

Aromasin® tablets of 25 mg strength are used. The daily dose is based on the outcome of the part 1 of the Phase I study.

A multicenter, placebo controlled, double blind randomized trial is organized recruiting 100 ISS (idiopathic short stature) boys in puberty (Tanner II-V), with growth potential (bone age <16 yrs), normal GH responses to stimuli (peak GH >10 ng/mL) and no other identifiable cause of short stature. Blood samples for the same parameters as study 1 are obtained as well as accurate anthropometric measures (stadiometer height, weight), DEXA of the lumbar spine and whole body with a tissue bar to assess body composition. Subjects are assigned to either placebo or Aromasin® at a dose selected from earlier studies. Bone age for determination of skeletal maturation and height prediction are obtained at baseline. The studies are conducted for 2 years and the subjects are followed until final height is achieved.

### EXAMPLE 3

A combination of Aromasin® and Genotropin® (recombinant human Growth Hormone) is given to boys with growth hormone deficiency. This is a multi-center trial with enrollment of at least 4 patients per group.

Aromasin® is given in a dose of from about 25 to about 50 mg/day.

Growth hormone is given as Genotropin® (recombinant human Growth Hormone) in a dose of from about 0.043 to about 0.1 mg/kg/day. As many as 3-4 arms are included in this trial; GH (either at one or two doses as below)+placebo; GH at standard dose (0.043 mg/kg/day)+Aromasin®, GH at “pubertal dose” approved by FDA (see, for example, Nutropin AQ PI) of 0.1 mg/kg/day+Aromasin®. End point is near final/final height.

The following references are noted in the application:

1. Morishima et al, J Clin Endocrinol Metabol 1995; 80:3689-3698

2. Smith et al, N Engl J Med 1994; 331(16):1056-1061.

3. Constantine et al, J Clin Endocrinol Metabol 1998; 83:1348-1357

4. Dombernovsky et al, J Clin Oncol 1996; 16: 453-461

5. Tönshoff et al, Br J Clin Pract 1996: supplement 85:34-37

6. Nunez et al, Reprod Toxicol 1996; 10(5): 373-377

7. Metzger and Korrigan, J Clin Endocrinol Metabol 1994; 79: 513-518

8. Kletter et al, JCEM 2; 4010, 1997

9. Grumbach et al, Horm. Res. (1998), 49(Suppl. 2), 41-57

10. Hintz et al, NEJM 1999, 340 (7) 502-7

11. Mauras et al, Pediatr Res 45:4: part 2 of 2, 1999, page 94A

The examples and specific or preferred embodiments set forth herein are illustrative in nature and are not intended to be limiting of the invention defined by the claims. Additional embodiments of the invention will be apparent to one of ordinary skill in the art in view of the present specification and are equally within the scope of the claims.

What is claimed is:

1. A method for treatment of growth failure in an individual, comprising administering to the individual an aromatase inhibitor.

2. A method according to claim 1, for improving the final height of the individual.

3. A method according to claim 1, for improving bone maturation and/or delay of puberty.

4. A method according to claim 1, further comprising administering Growth Hormone to the individual.

5. A method according to claim 4, wherein the Growth Hormone is human Growth Hormone.

6. A method according to claim 1, wherein the individual is a pre- or pubertal child.

7. A method according to claim 6, wherein the child is a boy with growth hormone deficiency (GHD).

8. A method according to claim 6, wherein the child is a boy with idiopathic short stature.

9. A method according to claim 1, wherein the aromatase inhibitor is exemestane.

10. A method according to claim 4, wherein the aromatase inhibitor and Growth Hormone are administered simultaneously.

11. A method according to claim 4, wherein the aromatase inhibitor and Growth Hormone are administered separately.

12. A method according to claim 4, wherein the aromatase inhibitor and Growth Hormone are administered sequentially.

13. Composition comprising an aromatase inhibitor and Growth Hormone.

14. Composition according to claim 13, wherein the aromatase inhibitor and Growth Hormone are included in amounts effective to improve final height in a growth failure therapy.

15. Composition according to claim 13 wherein the aromatase inhibitor is exemestane.

16. Composition according to claim 13 wherein the Growth Hormone is human Growth Hormone.
